(19) United States
(12) Patent Application Publication
(10) Pub. No.: US 2026/0165632 A1
(43) Pub. Date: Jun. 18, 2026

(54) CYCLE LENGTH MAPPING FOR TREATING ATRIAL FIBRILLATION

(71) Applicant: CoreMap, Inc., Burlington, MA (US)

(72) Inventor: Peter S. Spector, Colchester, VT (US)

(21) Appl. No.: 19/357,936

(22) Filed: Oct. 14, 2025

Related U.S. Application Data
(60) Provisional application No. 63/707,407, filed on Oct. 15, 2024.

(51) Int. Cl. A61B 5/367; A61B 5/287; A61B 5/339; A61B 5/346

(52) U.S. Cl. CPC A61B 5/367; A61B 5/287; A61B 5/339; A61B 5/346

(57) ABSTRACT

The invention provides methods for mapping the electrical activity of the heart and create cycle length maps to guide ablation to treat atrial fibrillation.

### BACKGROUND

Atrial fibrillation (AFib, or AF) is a common cardiac arrhythmia and a progressive disease that, if left untreated, worsens over time. Atrial fibrillation can lead to various problems such as blood clots, stroke or heart failure. Mechanisms of atrial fibrillation are discussed in Roney, 2020, Challenges associated with interpreting mechanisms of AF, Arrhythmia & Electrophysiology Rev 8(4):273-284, the contents of which are incorporated by reference for all purposes

Treatment of atrial fibrillation has involved mapping the cardiac substrate to identify regions for ablation. Some of those treatments have resulted in ambiguous outcomes. One issue is that in some cases of persistent atrial fibrillation, excitation patterns vary over time, a phenomenon that complicates mapping of activation times and potentially introduces errors when acquiring data sequentially. Substrate mapping generally refers to approaches that map tissue properties, rather than excitation patterns. Substrate mapping techniques to guide ablation may involve mapping the cardiac substrate to identify regions associated with complex fractionated atrial electrograms (CFAE), regions of the tissue from which electrograms are inconsistent. Other substrate mapping techniques have examined low voltage or dominant frequency as indicators to guide ablation. Nevertheless, ablation treatments to date have not always been fully successful.

### SUMMARY OF THE INVENTION

The present invention provides methods and systems useful for mapping cycle length, and cycle length variance, of waves of electrical activity in the cardiac substrate. It is thought that areas of the substrate associated with a short effective refractory period (the time after an action potential stimulus when the cell is unable to be stimulated again) correlate with areas of complex activation patterns, suggesting themselves as being implicated as drivers of atrial fibrillation. Logically, the effective refractory period of a cardiomyocyte limits the cycle length of that cell. As a corollary, the effective refractory period of a region of cardiac tissue can be estimated from a minimum cycle length measured during atrial fibrillation. The invention uses cardiac mapping systems and methods to measure electrical activity from various regions in the heart, outputting those measurements as electrograms. Those electrograms (EGMs) are subject to an annotation of characteristic deflections or detection operation (e.g., automated operations such as calculation of local maxima in first derivative of EGM as activation time). For the set of characteristic deflections of one EGM, an algorithm is applied to identify biophysiological lengths of cycles consistent with the observed characteristic deflections. The algorithm may involve, for each characteristic deflection, measuring time back to a number N of prior characteristic deflections. For that characteristic deflection, the algorithm may model a distribution of that set of “N back” putative cycle length measurements. For example, a kernel density estimation (KDE) may be made for all of the N-back putative cycle length measurements for one characteristic deflection. The peak of the KDE curve may be recorded as the cycle length for that characteristic deflection. By such operations, a cycle length is identified for each of the characteristic deflections annotated or detected from any one electrogram.

It is noted that the described operations provide a result for characteristic deflections whether governed by a constant or changing cycle length. Additionally, it is noted that in general one electrogram will be taken by measuring one location in cardiac tissue with an electrode of a mapping catheter, and thus cycle lengths are identified for each location in the cardiac tissue. Additionally, because each characteristic deflection is assigned a cycle length, a cycle length variability may be determined for each location in the cardiac tissue. Additionally, the described operations provide results regardless of what number of distinct activation waves are measured at the location in cardiac tissue (where two or more circuits send waves past one electrode, the operations will provide two or more corresponding measures of cycle length and cycle length variability).

The described operations may be performed on data gathered using a mapping catheter with numerous (e.g., dozens or more) of electrodes in a small (e.g., about 1 cm square) electrode array. What is output is useful as a substrate map, and the electrode array may be passed across regions of cardiac substrate to build the map up over multiple passes. Each electrode, on each pass, generates an electrogram for one region of the cardiac tissue. The described characteristic deflection detection and operation provide cycle length and cycle length variability data for each of those regions in the heart. Collectively, those data provide cycle length maps for the heart.

The cycle length maps are useful to characterize the substrate's refractory properties. Effective refractory periods may be estimated for regions of the cardiac tissue from the cycle length map. Reduced refractory periods are understood to result from atrial-fibrillation related remodeling and may be observed more frequently in patients with persistent atrial fibrillation. Thus cycle length mapping by systems and methods of the disclosure may be used to guide the treatment of atrial fibrillation (e.g., guide ablation). Due to the physiology of cardiac propagation, faster regions (longer cycle length) typically drive slower regions. This relates to the spread of information through propagating waves of excitation in the heart. Faster sources send their waves towards slower sources progressively sooner with each beat and hence their waves are able to reach and influence the slower sources while slower sources cannot reach the faster sources. The overall result is that faster regions have a higher probability of being AF sources than slower regions. Stationary drivers are organized (stable cycle length) but fibrillation, globally, is disorganized (varying cycle length and directionality). As a result, if a region is seen to have a high degree of organization it is likely close to a stationary driver. The complex propagation of AF causes organization to diminish within a short distance from a stationary source. Thus, the identification of local organization during fibrillation defines a site as having a high probability of being in close proximity to a stationary driver. Importantly, the interpretation of this feature is independent of surrounding spatial context (other than global disorganization (i.e. AF)); and, as such, this is a metric that is only vulnerable to sequential sampling error by virtue of a potential for false negatives, but not false positives.

In certain aspects, the invention provides methods for cycle length mapping. Methods may include recording electrical activity at a location on cardiac tissue as an electrogram; identifying an characteristic deflection within the electrogram; measuring times from the characteristic deflection to previous characteristic deflections; modeling a distribution of the measured times; and assigning a peak value of the distribution as a cycle length associated with the characteristic deflection at the location on the cardiac tissue. The measuring step may include measuring back in time, on the electrogram, from the characteristic deflection, to a pre-determined number N of previous characteristic deflections on the electrogram. The modeled distribution may include a kernel density estimation (KDE). In addition, methods of the invention specifically identifies sites of endo-epicardial dissociation that contribute to AF perpetuation.

The method may include: identifying multiple characteristic deflections in each of multiple electrograms taken from corresponding locations on the cardiac tissue; and repeating the measuring, modeling, and assignment steps for the multiple characteristic deflections to create a cycle length map representing cycle lengths at the locations on the cardiac tissue. In some embodiments, the cycle length map further presents cycle length variability at the locations on the cardiac tissue. The method may further include estimating, from the cycle length map, an effective refractory period for one or more of the locations on the cardiac tissue.

In certain embodiments, the recording step includes inserting a cardiac mapping catheter into a chamber of a heart of a patient and storing electrograms captured using a plurality of electrodes of an electrode array disposed on a distal portion of the catheter. The method may include receiving signals from the electrode array at a computer system connected to the catheter, and operating the computer system to create and store the cycle length map. The plurality of electrodes may be in the electrode array as pairs of electrodes, each pair in a stack that is substantially orthogonal to a surface of the cardiac tissue during use, e.g., optionally in a unipolar, close, orthogonal (OCU) configuration.

Preferably, the method includes displaying a representation of the heart with the effective refractory period for the one or more of the locations on the cardiac tissue overlaid on the representation of the heart.

Related aspects may include systems for cycle length mapping. Systems may include a cardiac mapping catheter with an electrode array disposed on a distal portion of the catheter, the electrode array comprising a plurality of electrodes, and a computer system operably connectable to the catheter. The computer system includes a processor coupled to a memory subsystem having instructions stored therein executable by the processor to operate the electrode array to record electrical activity at a location on cardiac tissue as an electrogram when the distal portion of the catheter is inserted into a chamber of a heart of a patient. The computer system is further operable to (i) identify an characteristic deflection within the electrogram; measure times from the characteristic deflection to previous characteristic deflections; model a distribution (such as a KDE) of the measured times and (ii) assign a peak value of the distribution as a cycle length associated with the characteristic deflection at the location on the cardiac tissue.

In some embodiments, the computer system measures, back in time from the characteristic deflection, to a pre-determined number N of previous characteristic deflections on the electrogram. The computer system may be operable to: (i) identify multiple characteristic deflections in each of multiple electrograms taken from corresponding locations on the cardiac tissue; and (ii) repeat the measuring, modeling, and assignment steps for the multiple characteristic deflections to create a cycle length map representing cycle lengths at the locations on the cardiac tissue. The cycle length map may include cycle length variability at the locations on the cardiac tissue. In one instance, a transmission site (one that is in close proximity to a driver or at a site where directionality is constrained) may have a consistent directionality but a varying cycle length but an origin site (i.e., that is a stationary driver) will have both consistent directionality and organized cycle length and that combination is a valuable feature of the map. In certain embodiments, the computer system is operable to estimate, from the cycle length map, an effective refractory period for one or more of the locations on the cardiac tissue.

Preferably, the memory subsystem stores therein electrograms captured using the plurality of electrodes of the catheter. The system may include a processing device (e.g., as a bed-side kiosk) operably connected between the catheter and the computer system, wherein the processing device includes a at least one microprocessor that functions as a multiplexer, amplifier, or analog-to-digital (ADC) converter, wherein the processing device is a bedside instrument wired to the catheter that transmits data to the computer system. The plurality of electrodes may in the electrode array as pairs of electrodes, each pair in a stack that is substantially orthogonal to a surface of the cardiac tissue during use, e.g., with an OCU configuration. The computer system may be operable to display a representation of the heart with the effective refractory period for the one or more of the locations on the cardiac tissue overlaid on the representation of the heart.

### BRIEF DESCRIPTION OF DRAWINGS

FIG. 1 shows a system for mapping arrythmia or fibrillation cycle length.

FIG. 2 gives a close-up view of the head carrying an electrode array.

FIG. 3 is a detail view of an electrode.

FIG. 4 shows a plurality of electrograms (ECM).

FIG. 5 illustrates the measurement of times of prior characteristic deflections.

FIG. 6 shows a distribution of measured times between characteristic deflections.

FIG. 7 shows a median cycle length.

FIG. 8 shows a cycle length variability.

FIG. 9 is a cycle length map.

FIG. 10 is a cycle length variance map.

FIG. 11 shows a cycle length map and a cycle length variance map on a live display.

### DETAILED DESCRIPTION

The invention provides systems and methods that use intracardiac electrograms to map tissue substrate and specifically to map cycle length and cycle length variability at specific locations of the cardiac tissue. Systems and methods of the invention involve obtaining electrograms (ECM) that have been annotated or analyzed to identify characteristic deflections and then algorithmically determining cycle lengths for the characteristic deflections on an ECM that are consistent with those characteristic deflections and cardiac physiology. Cycle length mapping algorithms of the disclosure address an issue by which characteristic deflections that appear on an ECM may arise from diverse cycles or waves that propagate through the tissue so that each consecutive characteristic deflection in time is not a result of the same cycle (or rhythm or beat or circuit). Any suitable algorithms may be applied. Some embodiments use an “N-back” algorithm that, for each characteristic deflection, measures times back to a preceding number N of characteristic deflections. For the resulting set of measurements, a distribution of those measurements is modeled. A peak in the distribution may be taken as the cycle length associated with that characteristic deflection. The measuring and modeling may be performed for each characteristic deflection on the ECM to identify a set of one or more cycle lengths characteristic of a location in the cardiac substrate from which that ECM was generate, i.e., a location at which one electrode of a mapping apparatus made a measurement for a period of time.

Because the ECM is a measurement of electrical activity over time, and because characteristic deflections are identified over the ECM, and because characteristic cycle lengths may be assigned to each characteristic deflection, the disclosed operations may show a cycle length that is changing, over time, at a location on the substrate. Because the ECMs may be captured at multiple locations over the cardiac substrate, the output of the operations may be a map of cycle lengths, and cycle length variability, over the area of the cardiac tissue substrate. Accordingly, systems and methods of the disclosure apply operations such as an N-back algorithm for cycle mapping. Systems and methods of the disclosure provide for the use of those algorithms to assign features on electrograms to beats. Systems and methods of the disclosure may also include features useful to mask out variance and noise, and to give users tools (e.g., “sliders”) for weighting inputs, and other features described herein. For example, the N-back and cycle length mapping may include tools and operations that measure and determine confidence during mapping, which is valuable when making decisions from data that are inherently noisy and ambiguous.

Systems and methods of the disclosure include a mapping catheter used to map cardiac electrical activity. Any suitable cardiac mapping catheter may be used. A catheter of the disclosure may be used to detect cardiac electrical signals via one or more electrodes of the catheter and create a map of cycle lengths characteristic of arrythmia or fibrillation. Mechanisms of atrial fibrillation are discussed in Roney, 2020, Challenges associated with interpreting mechanisms of AF, Arrhythmia & Electrophysiology Rev 8(4):273-284, the contents of which are incorporated by reference for all purposes.

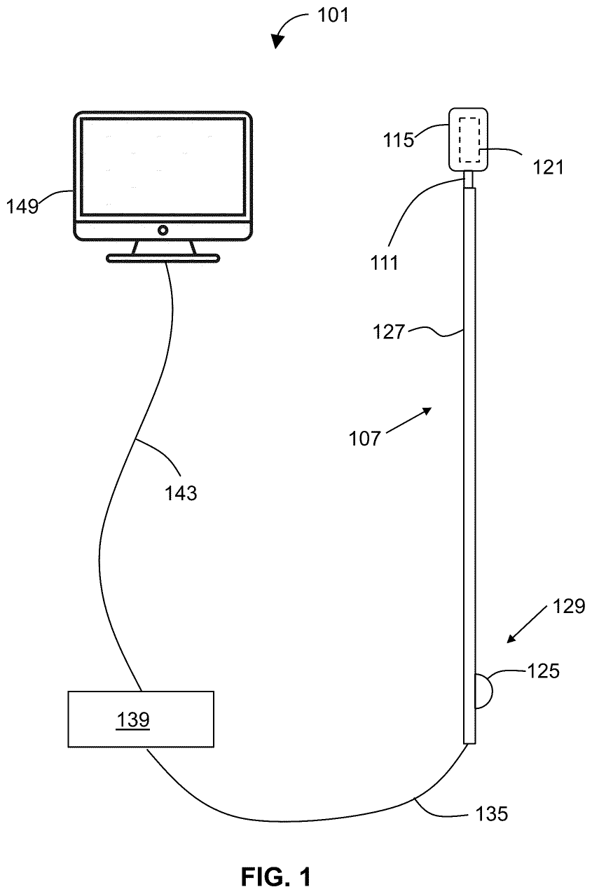

FIG. 1 shows a system 101 for mapping arrythmia or fibrillation. The system 101 includes a cardiac mapping catheter 107 of the invention. The cardiac mapping catheter 107 may generally include a shaft 111 extending to a head 115 carrying an electrode array 121. Electrodes of the electrode array 121 may optionally be connected via leads or at least one signal wire 135 to an optional processing device 139, which in turn have a data connection 143 to an optional computer system 149. The extend shaft 111 may extend through, and out of a distal end of, an introducer sheath 127. The catheter 107 may include a deformation mechanism 129 that is operable to change a shape, position, or dimension of the head 115 to thereby position the electrode array 121 at substantially any position within the heart. Any suitable deformation mechanism 129 may be included. Preferred embodiments use a switch, controller, or actuator 125 positioned at, or communicatively coupled via, a proximal end of the shaft 111 or sheath 127, to be manipulated by a surgeon from outside of a body of a patient during use of electrode array 121 in the heart.

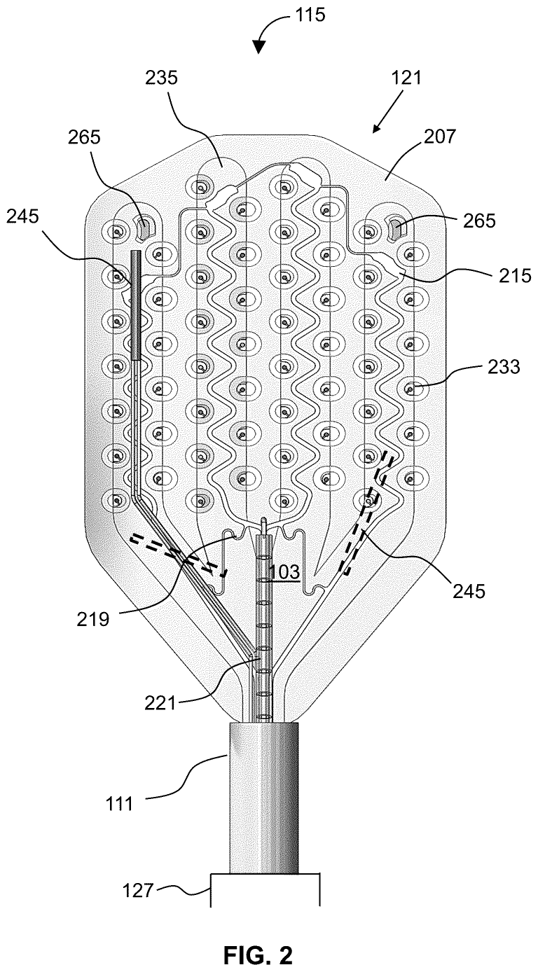

FIG. 2 gives a close-up view of the head 115 carrying an electrode array 121. In some embodiments, the head 115 includes a flexible support frame 215 extending within a deformable leaf 207 of a material, presenting the electrode array 121 on a tissue-facing surface. Due to the support frame 215, the head 115 may be substantially furled (or curled) closed, e.g., in a cylindrical configuration when the head is retracted within a sheath. When the head is extended out, in a distal direction, from a distal end of the sheath 127, the deformable leaf 207 of a material may assume an open configuration for mapping tissue.

The electrode array 121 generally includes a plurality of sensing electrodes 233 available on at least a tissue-facing surface of the deformable leaf 207 (optionally equally available on both surface of the leaf 207). The support frame 215 may be made with shape memory and super-elastic material such as Nitinol, or other alloys of nickel and titanium. The support frame 215 may be designed so that when the head 115 within a sheath, the deformable leaf 207 is substantially s-shaped, rolled, or folded. After the electrode array is deployed from a sheath, it opens up to an unfurled configuration such that the electrode array 121 lies against the tissue upon which it is deployed to match the contours of the tissue.

The deformable leaf 107 may include one or more layers of a suitable non-metallic material such as polyimide, polyether ether ketone (PEEK, or nylon, optionally with a first layer having a higher durometer number than a second layer material, such as a thermoplastic polymer e.g., the polyamide and polyether block polymer sold under the trademark PEBAX by Arkema Global (Colombes, France). In another example, the deformable surface may have a first layer with, for example, PEEK or a thermoplastic polyurethane having a durometer number of 87 D, and a second layer of a polyamide and polyether block polymer having a durometer number of 55 D. The second layer material may then form a web around the first material. In some embodiments, the second layer may form a web-like structure around the first layer. The web structure may appear as open interstices or may resemble a web, netlike, or netted pattern.

The support frame 215 may be connected to the deformable leaf 107 on either side of the deformable leaf 107, or it may be retained within the deformable surface. In some embodiments, the cardiac mapping catheter comprises a deformable surface such that the catheter is undulated upon deployment from a sheath and lies flat when deployed against tissue.

The head 115 may include a plurality of tines 235 or splines extending from the catheter shaft through the electrode array. For example, the tines 235 may appear as arms or in the form of slender projecting or branching pieces to engage the tissue surface. Each electrode 233 may be positioned along an edge of a tine 235, distributed over the head to form the electrode array 121.

The catheter 107 may include a user-controllable deformation mechanism 129 that is operable to deform the head 115. Any suitable deformation mechanism 129 may be included. Preferred embodiments use a first tension write 219 and a second tension wire 219 (not visible on the head 115, being on the reverse side). The tension wire 1219 may extend the length of the catheter body to the actuator 125. The wire 219 may be controllable and may be used to deform the surface of the catheter to match the surface of the cardiac tissue. By actively modifying the angle of the electrode array via the tension wires 219, the deformable leaf 107 may be bent in two different directions. In some embodiments, cardiac mapping catheters of the invention include a spine tube 221 in connection with the flexible support frame 215 of the electrode array 121 and a pull wire 219 also in connection with the flexible support frame. In some embodiments, the spine tube 221 is connected to the catheter shaft 101 and the tension wires 219, and housed within or upon the deformable leaf 107. The spine tube 221 may be notched to aid in deflection of the electrode array 121 once the array is deployed from a sheath. The tension wires 219 may be provided so that when the electrode array 121 is deployed out of a sheath 127, the tension wires 219 allows the electrode array to deflect in either direction such that the physician can manipulate the electrode array to a desired position upon the tissue.

Catheters of the invention may include one or more location or position markers 245. Each position marker 245 may be a magnetic coils, (e.g., as magnetic tracking sensors or magnetic location sensors). The position markers 245 may be positioned on opposing sides and/or opposite ends of the deformable leaf 107. Having magnetic coils in the opposing positions allow for shape detection of the array and identification of each electrode's position in 3D space. The position markers 245 may provide a 5 or 6 degrees of freedom (DOF) sensor, wherein the degrees of freedom describe the number of axes in which a rigid body moves freely in 3D space. In some embodiments, data from the magnetic sensors is used to calculate the position of the electrode array 121 to model a shape of a surface from which measurements are made.

In some embodiments, the electrode array may further comprise a plurality of pacing electrodes 265, positioned for the measurement of a refractory period and/or to stimulate activation of a cardiac rhythm. Thus, in some embodiments of the invention, the cardiac mapping catheters of the invention allow for sensing, mapping, and pacing functions on the single device.

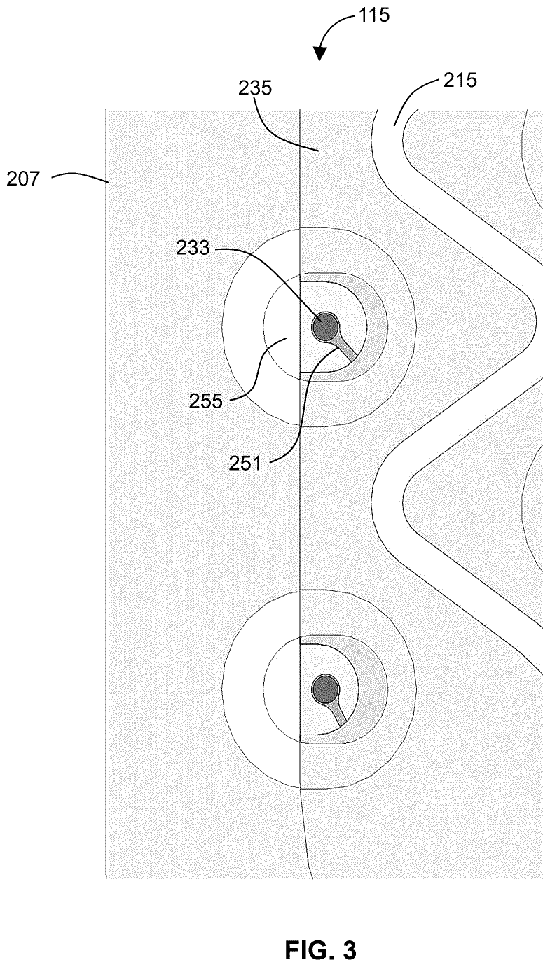

FIG. 3 is a detail view of an electrode 233 of the electrode array 121, here viewed from a tissue-facing (or “posterior”) surface of the deformable leaf 207. In optional embodiments, both surfaces of the leaf 207 are tissue-facing; the electrodes are exposed on both sides; and position and directionality are determined using the position markers 245 and/or in software. Portions of the support frame 215 and at least one edge of one tine 235 are visible. Preferably, the electrodes 233 are present in an ordered array, with pairs of the electrodes 233 being separated by a known distance. Each electrode 233 as shown may, in fact, be a single electrode, or each electrode as shown may include a pair of electrode contact elements that are stacked. Each electrode 233 may comprise two or more materials. For example, the electrodes may include an iridium oxide coating 203 surrounded by a layer or region of insulating material 255.

In some embodiments, individual sensing electrodes 133 include a copper trace 251 and a copper pad that may be plated with gold. The gold may cover the copper components of the electrode to prevent the copper from leaching into solution. The electrode may then be further coated with iridium oxide to provide a layer of iridium oxide to reduce the impedance and to improve signal-to-noise ratio. In some embodiments, the electrode comprises a slot below the insulating layer into which the copper trace is embedded such that the gold plating is on top of the copper electrode.

Each electrode 233 may include electrode contact elements shaped substantially as thin discs. The electrodes, as thin discs, may be positioned as a contiguous array on the surface of the deformable surface. The electrodes may be positioned along an edge of a tine 235 or spline. Notably, the height, or alternatively the thickness, of the electrode may be less than about 150 μm. In some embodiments, the height of the electrode is less than about 25 μm. Height, as used herein, means the height of the electrode as measured from the bottom of the electrode relative to its orthogonal position on the tissue to the top of the electrode. Alternatively, the height of the electrode may also refer to the thickness of the electrode as a thin disc. In some embodiments, the leaf 207 has a plurality of openings to allow biological fluid and/or a wash fluid such as saline to pass through the head 115.

In non-limiting embodiments, the diameter of each electrode 233 may be 150 μm such that the electrode surface area is less than or equal to 0.018 mm\hat{ }2. The electrode array may comprise a number (e.g., 30, 40, 44, 50, 56 . . . ) electrodes on each side of the deformable surface. The electrode array width may be less than or equal to 15 mm, with an overall length less than or equal to 26 mm. The array sensing area may be less than or equal to 15 mm\hat{ }2. The cardiac mapping catheter may be used with a sheath 127, such as an 8.5 F steerable sheath with a diameter ranging from about 0.122 inches to about 2.83 mm.

Electrodes may be unipolar, bipolar, or of a hybrid arrangement involving a stacked pair of bipolar electrodes that provides advantages of unipolar electrodes. For conventional electrophysiological procedures, intra-cardiac electrodes have been deployed in two distinct configurations, unipolar and bipolar. For bipolar recordings, a different electrode is positioned at the site of interest while the indifferent electrode is positioned close to, but spaced away from, the different one. Wave fronts that pass the electrodes induce similar signals at both poles, but with the signals being shifted in time. In the unipolar recording mode, a different electrode is positioned at the site where the electrical potential must be determined while the indifferent electrode is positioned a large distance from the heart, at zero potential. Unipolar electrodes are adequate but have a tendency to include far field electrical activity in the recorded signal which can result in a fractionated electrogram, of concern when trying to map complex arrhythmias (e.g. cardiac fibrillation) and accurately identifying local activation time. Bipolar electrode configurations minimize that fractionation by placing both electrodes within the heart at a relatively narrow distance apart. Both electrodes of a bipolar pair measure approximately the same far field electrical activity with the result that the resultant electrogram includes little far field signal. Here, catheters of the disclosure may use another electrode configuration comprising pairs of electrodes that are, in fact, bipolar but in which the pair is designed to sit in a stack over one location of the tissue so that, like with unipolar electrodes, signal recorded by the stacked electrode pair tends to reflect electrical activity from all regions of the heart even when the stacked electrode pair is placed directly over a particular region. Thus, each electrode may be provided as, or as part of, an electrode pair having a “stacked” or orthogonal close unipolar (OCU) electrode configuration. This electrode array may include a two dimensional array of stacked electrode pairs (e.g., first electrode and second electrode layered with insulator therebetween). The first electrode is configured to be close to a location on a surface of target tissue and the second electrode is separated from the first electrode but over the same location. Each electrode pair may be provided in an orthogonal, close, unipolar (OCU) configuration. Of the pair, the two electrodes are stacked such that when the leaf 207 is against tissue, an axis with rotational symmetry through the stack of the two electrodes is substantially orthogonal to a surface of the tissue where the electrode pair is making a reading (“orthogonal” nature of the pair). A distance between the first and second electrodes is preferably about a same order of magnitude as a diameter or thickness of each electrodes so that the members of the pair are close to each other (“close” nature of the pair). The one pair of bipolar electrodes may operate to measure electrical activity from all regions of the heart as expected from unipolar electrodes (“unipolar” nature of the pair).

Such a configuration, referred to as an OCU configuration, addresses the limitations of existing unipolar and bipolar electrodes. In particular, recorded electrical potential of current bipolar electrodes vary with their orientation relative to the direction of a passing wavefront. Additionally, because bipolar electrodes have both electrodes on a given surface, there is potential inclusion of distinctly different electrical activity from each electrode. As such, by providing electrodes oriented perpendicular to the tissue plane, via the orthogonal close unipolar (OCU) design of the present invention, the electrode array of the present disclosure retains the superior near/far-field discrimination of common bipolar electrode recordings with the directional independence and smaller footprint of unipolar recordings. Furthermore, the unipolar electrode configuration of the present invention retains all of the spatial resolution benefits of a contact bipolar configuration, but with the additional spatial resolution enhancement conferred by a smaller footprint.

Embodiments of the disclosed cardiac mapping catheter may include unipolar or bipolar electrode configurations. Preferred embodiments include OCU configurations. The electrodes 233 preferably form an electrode array on the head 115 connected to the distal portion of the catheter 107, in which the catheter includes a deformation mechanism operable to deform the distal portion to position an electrode 233 of the electrode array at substantially any location on a cardiac surface

The mapping catheter 107 is used to map electrical activity of the heart. The cycles of the heart are determined by The Cardiac Conduction System (CCS, also called the conduction system of the heart), which is the main driver of proper heart function. The CCS transmits the signals generated by the sinoatrial node—the heart's pacemaker, to cause the heart muscle to contract, and pump blood through the body's circulatory system. The pacemaker signal travels through the right atrium to the atrioventricular node, along the bundle of His, and through the bundle branches to Purkinje fibers in the walls of the ventricles. The Purkinje fibers transmit the signals more rapidly to stimulate contraction of the ventricles.

Here, the catheter 107 is operated in a chamber of the heart. Each electrode 233 generates an electrogram (ECM), typically stored within and/or viewable on the connected computer system 149. The computer system 149 may be operable to analyze the cardiac electrical signals with an algorithm to locate the source of atrial fibrillation in a patient. The invention provides methods for mapping the electrical activity of the heart, specifically cycle length mapping, to guide cardiac ablation. Electrical activity is measured with electrodes and activation characteristic deflections within the electrical activity are recorded. Once the activation characteristic deflections are detected time between them is measured and a distribution of the characteristic deflections is modeled and all peak values are found. The activations are then fitted to the peak values to determine an underlying frequency of activation. This determines the cycle length and helps to create a cardiac map.

Characteristic deflections on an ECM may be provided to the mapping operations by annotation or characteristic deflection detection operations. In some embodiments, a cardiac physiologist manually annotates characteristic deflections on an ECM (e.g., on a computer screen). In certain embodiments, the computer system 139 performs an characteristic deflection detection operation.

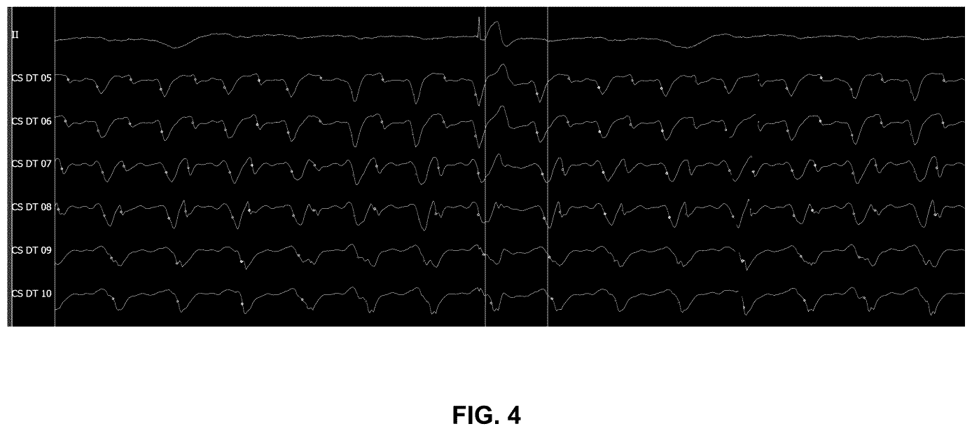

FIG. 4 shows a plurality of electrograms (ECM) recorded from a corresponding plurality of electrodes 233 of a catheter 107. As shown, points have been drawn on each ECM to represent characteristic deflections. As noted, a user may annotate the ECM to identify characteristic deflections. Characteristic deflection detection may be performed automatically by the computer system 149. Detection is discussed in Osorio, 2022, An efficient hybrid methodology for local activation waves detection under complex fractionated atrial electrograms of atrial fibrillation, Sensors 22(14):5345, the contents of which are incorporated by reference for all purposes. Preferred embodiments use an characteristic deflection detection algorithm performed by the computer system 149.

On computer-implemental characteristic deflection detection operation includes: (i) detecting all downstrokes; (ii) filter away detected downstrokes that are insignificant due to noise leaving filtered downstrokes (that are understood to be significant); (iii) classifying each filtered downstroke (e.g., as atrial, pacing, or ventricularfar type); (iv) removing, from the classified filtered downstrokes, ventricular farfield downstrokes, leaving atrial activation downstrokes; and (v) aggregating the atrial activation downstrokes into characteristic deflections. As used herein, ventricularfar is a label for a category of ventricular, far-field downstroke signals. Those steps may be implemented in software. Briefly, each step would be coded to perform the following operations.

First, (i) all downstrokes are detected. The computer system finds a timestamp for each positive or negative peak (preferably including flat peaks no longer than 20 ms; preferably setting a maximal allowed downstroke duration of 30 ms). Adjacent downstrokes that do not meet a criteria for relative prominence (e.g., time between ends of first and start of second <5 ms) are combined). The criteria and the values may be varied. The output will be timestamps for the start and end of each downstroke.

Second, (ii) detected downstrokes that are insignificant due to noise are filtered away leaving filtered downstrokes (that are understood to be significant). Any suitable criteria may be used such as an amplitude threshold etc.

Third, (iii) each filtered downstroke is classified (e.g., as atrial, pacing, or ventricularfar type). For each filtered downstroke, a set of characteristics may be calculated. Preferred characteristics include: peak-to-peak amplitude; max slope; slope-weighted time; or others. Classification may be made with reference to pacing windows, e.g., “pacing” if downstroke overlaps with pace blanking window; “ventricularfar” if overlap with ventricular blanking window; “atrial” if criteria for “pacing” or “ventricularfar” are not met. The output is classified, filtered downstrokes.

Fourth, (iv) ventricular farfield downstrokes are removed from the classified filtered downstrokes, leaving atrial activation downstrokes.

Fifth, (v) the atrial activation downstrokes are aggregated into characteristic deflections. A characteristic deflection can be consistent of a single temporally isolated downstroke or multiple closely timed downstrokes of the same type. In the case of an characteristic deflection consists of multiple downstrokes, the characteristic deflection local activation time (LAT) is calculated based on the slope-weighted time, amplitude, and slope of the downstrokes that make up the characteristic deflection. To aggregate, any adjacent downstrokes may be provisionally placed in one segment mask (a conceptual construct by which the computer system 149 holds adjacent downstrokes together to analyze for aggregation). For each segment mask, the computer system 149 calculates the contribution of each downstroke to the aggregated characteristic deflection LAT (downstrokes that contribute less than 0.5 ms to the aggregated characteristic deflection LAT are deemed insignificant and excluded), and build an characteristic deflection using remaining downstrokes that are less than 20 ms apart. The final aggregated characteristic deflection LAT is the last aggregated LAT that satisfies the following condition: |aggregatedLATn-1−aggregatedLATn|>=0.5 ms. The output of this fifth (v) step is a set of characteristic deflections with any of the following attributes: (1) LAT, (2) Confidence=max(downstroke scores), (3) Characteristic deflection mask: start and end timestamps, (4) Peak-to-peak amplitude=max(downstroke peak-to-peak amplitudes), (5) a fractionation score, and (6) Type: Inherited from downstroke type (atrial, ventricularFar, pacing). Once the activation characteristic deflections have been identified, the time between each activation will be measured.

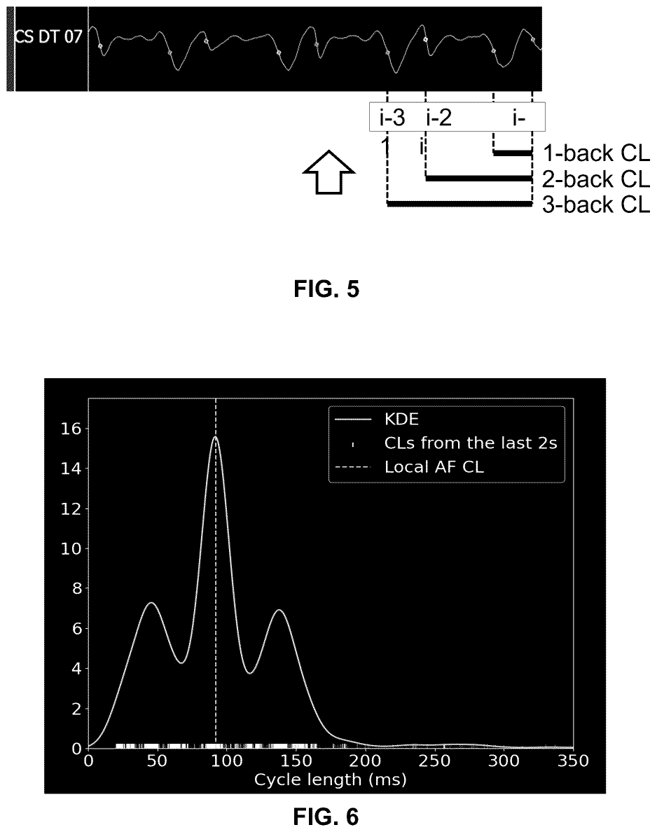

FIG. 5 illustrates the measurement of 3-back times to prior characteristic deflections. Following the measurement of activation characteristic deflections and identification of the time in between them, the activation characteristic deflections will be modeled.

FIG. 6 shows the modeling of a distribution of measured times between characteristic deflections. The modeled distribution may be in the form of a Kernel Density Estimation (KDE). A KDE is the application of kernel smoothing (a type of weighted moving average wherein the closer points are given higher weights) for probability density estimation, i.e., a non-parametric method to estimate the probability density function of a random variable based on kernels as weights. The KDE is discussed in Nagy, 2022, Cycle length evaluation in persistent atrial fibrillation using kernel density estimation to identify transient and stable rapid atrial activity, Cardiovascular Eng Tech 13(2):219-233, the contents of which are incorporated by reference for all purposes. The KDE addresses data smoothing problems where inferences about the population are made based on a finite data sample. Here, the peak activation characteristic deflection of the KDE may be taken as the cycle length indicative of local atrial fibrillation.

The highest peak value of the KDE may be taken to be a cycle length of an characteristic deflection at a location in the heart where an electrode captured to electrogram.

The modeled distribution and activation characteristic deflections disclosed herein may be displayed on a display device of the computer system 149. The display may be any piece of suitable equipment such as a computer screen, a tablet, an external monitor, or any other piece of equipment capable of producing a visible picture. The virtual display may further comprise a computer system to process data. The computer system may optionally include additional hardware to assist with data processing such as the central processing unit (CPU), random access memory (RAM), a motherboard, computer data storage system, a graphics card, a sound card, and/or computer case. This may also include extra external devices to assist the user such as a mouse, a keyboard, and/or speakers. Additionally, software to assist in cardiac mapping may be installed on any relevant portions of hardware. The computer system may also be configured to pair to Wi-fi, Bluetooth, or any other hospital databases and/or record systems.

The computer system 139 performs the described characteristic deflection detection operation, and N-back and KDE algorithms to assign cycle length and cycle length variability to a location on the cardiac substrate from which an electrode recorded an ECM.

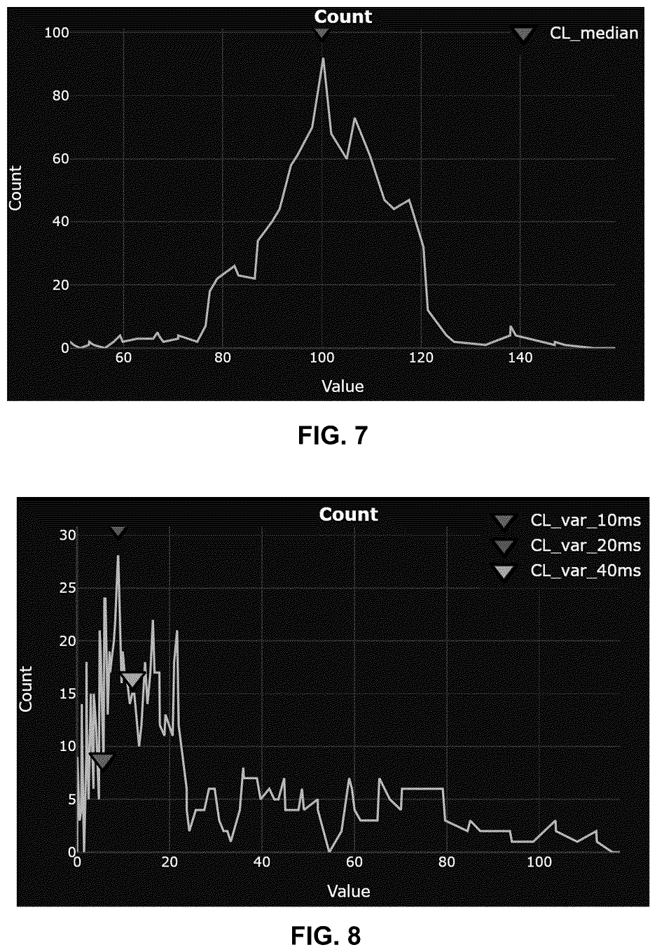

FIG. 7 shows a median cycle length that the computer system assigns to a location.

FIG. 8 shows a cycle length variability assigned to the location.

Because the catheter 107 has an electrode array 121 with a plurality of electrodes 233 and may be moved about a chamber of the heart to generate ECMs for a plurality of distinct locations across the tissue substrate, and because the computer system may determine cycle length and cycle length variability of each of those locations, and because the catheter includes position markers 245, the computer system can build a 3D representation of the heart and also build maps of cycle length and cycle length variability that may be displayed as an overlay (or color map or other suitable graphic format) on the 3D representation of the heart.

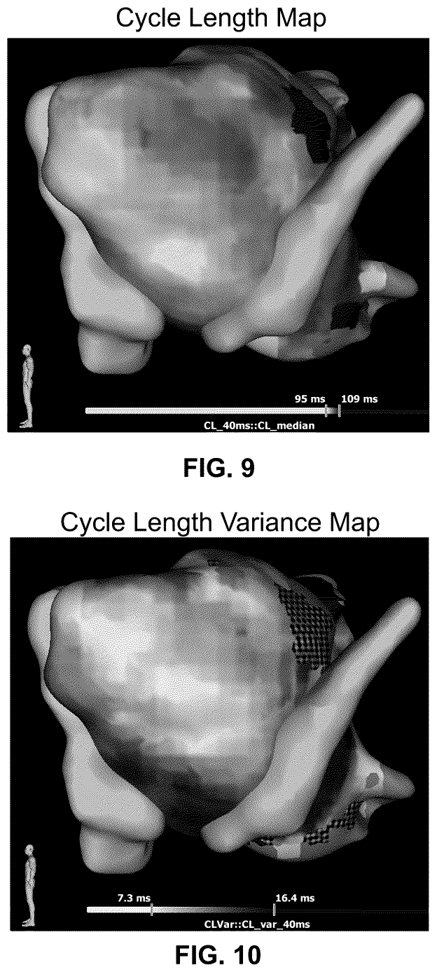

FIG. 9 is a cycle length map as displayed or provided by the computer system 149 superimposed on a 3D representation of the heart. It is noted that a cycle length map is a valuable output that contains a wealth of clinically-actionable information. A cycle length or cycle length variance map of the invention need not be displayed on an electronic display such as a monitor. For example, it may be clinically valuable to have the computer system 149 create such a map and store the map(s) in memory for future reference or for export to another medical device's coordinate tracking system. A cycle length or cycle length variance map may be written to a patient's electronic medical records, and/or may be provided in print (e.g., and stored in a file.

FIG. 10 is a cycle length variance map as displayed or provided by the computer system 149 superimposed on a 3D representation of the heart. As noted, the map(s) may be displayed or stored. In certain preferred embodiments, the cycle length and/or the cycle length variance map are displayed live, in real-time, during an intracardiac catheterization procedure.

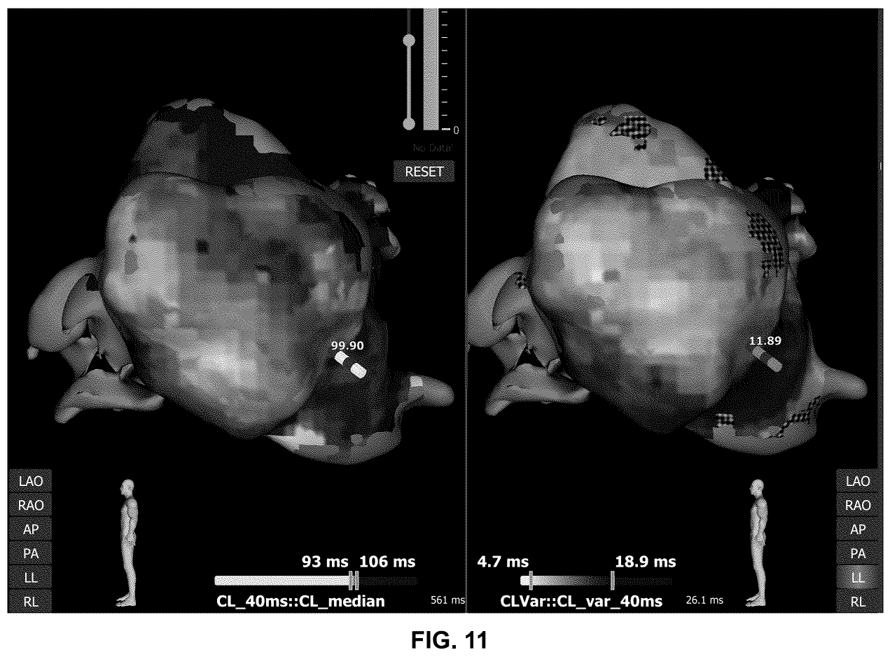

FIG. 11 shows a cycle length map (left panel) and a cycle length variance map (right panel) as those would be displayed live (e.g., on a computer monitor in an electrophysiology cath lab) during an intracardiac catheterization procedure such as a mapping procedure or even during an ablation procedure. The maps are useful for guiding ablation.

It is understood that cycle length and cycle length variance provide information about refractory properties such as the effective refractory period of the mapped locations of the heart.

Reduced refractory periods are understood to result from atrial-fibrillation related remodeling and may be observed more frequently in patients with persistent atrial fibrillation. Thus cycle length maps as displayed by systems and methods of the disclosure may be used to guide the treatment by ablation of atrial fibrillation. Cycle length and refractory period are discussed in Unger, 2021, Cycle length statistics during human atrial fibrillation reveal refractory properties of the underlying substrate: a combined in silico and clinical test of concept study, Europace 23:i133-i142, the contents of which are incorporated by reference for all purposes. The cycle length (left panel) and a cycle length variance map (right panel) that are shown represent what would be displayed live, in real-time, by the computer system 149. A surgeon may rotate the display, or may continue the mapping operations with the catheter (e.g., continue data collection with the electrode array 121) to have the displayed maps continuously updated, or “built up”, as greater areas of the heart are mapped.

With reference to the cycle length map (left panel) and the cycle length variance map (right panel) that may be shown on a live display, systems and methods of the disclosure may also include features useful to mask out variance and noise, and to give users tools (e.g., “sliders”) for weighting inputs, and other features described herein. For example, any of the thresholds or measure of the described characteristic deflection-detection operations may be presented to a user as adjustable criteria. The user can change a value (e.g., drag a slider on a touch-screen to reset a parameter of downstroke detection or any other step). Example of user-tunable variables that may be adjusted on the fly include maximal flat-peak inclusion (e.g., move between 10 and 30 ms) or critical prominence for downstroke (e.g., time between ends of first and start of second < a value between 2 and 10 ms). Within the characteristic deflection detection, N-back, or cycle length mapping, any value or criteria may be provided with controls by which the user may optimize, measure, and/or determine confidence during mapping.

As discussed above, cycle length maps and/or cycle length variance maps may be provided by analysis of EGMs from intracardiac mapping catheters. However, cardiac electrical activity may be measured by any other suitable test or method.

Heart activation data may also be gathered via an echocardiogram. An echocardiogram is the visual imaged formed by echocardiography (also known as a cardiac ultrasound). Echocardiography is routinely used in the diagnosis, management, and follow-up of patients with any suspected or known heart diseases. It can provide a wealth of helpful information, including the size and shape of the heart (internal chamber size quantification), pumping capacity, location and extent of any tissue damage, and assessment of valves. An echocardiogram can also give physicians other estimates of heart function, such as a calculation of the cardiac output, ejection fraction, and diastolic function (how well the heart relaxes). Echocardiograms are a noninvasive procedure; they use ultrasound and which does not break the skin or enter the body.

There are three primary types of echocardiography: transthoracic, transesophageal, and intracardiac. A standard echocardiogram is also known as a transthoracic echocardiogram (TTE) or cardiac ultrasound, and it is used for rapid evaluation of a patient at their bedside. In this case, the echocardiography transducer (or probe) is placed on the chest wall (or thorax) of the subject, and images are taken through the chest wall. This is a non-invasive, highly accurate, and quick assessment of the overall function of the heart.

TTE utilizes several “windows” to image the heart from different perspectives. Each window has advantages and disadvantages for viewing specific structures within the heart and, typically, numerous windows are utilized within the same study to fully assess the heart. Parasternal long and parasternal short axis windows are taken next to the sternum, the apical two/three/four chamber windows are taken from the apex of the heart (lower left side), and the subcostal window is taken from underneath the edge of the last rib.

TTE utilizes one- (“M mode”), two-, and three-dimensional ultrasound (time is implicit and not included) from the different windows. These can be combined with pulse wave or continuous wave Doppler to visualize the velocity of blood flow and structure movements. Images can be enhanced with “contrast” that are typically some sort of micro bubble suspension that reflect the ultrasound waves.

A transesophageal echocardiogram (TEE) is an alternative way to perform an echocardiogram. A specialized probe containing an ultrasound transducer at its tip is passed into the patient's esophagus via the mouth, allowing image and Doppler evaluation from a location directly behind the heart. It is most often used when transthoracic images are suboptimal and when a clearer and more precise image is needed for assessment. This test is performed in the presence of a cardiologist, anesthesiologist, registered nurse, and ultrasound technologist. Conscious sedation and/or localized numbing medication may be used to make the patient more comfortable during the procedure.

TEE, unlike TTE, does not have discrete “windows” to view the heart. The entire esophagus and stomach can be utilized, and the probe advanced or removed along this dimension to alter the perspective on the heart. Most probes include the ability to deflect the tip of the probe in one or two dimensions to further refine the perspective of the heart. Additionally, the ultrasound crystal is often a two-dimension crystal and the ultrasound plane being used can be rotated electronically to permit an additional dimension to optimize views of the heart structures. Often, movement in all of these dimensions is needed.

TEE can be used as stand-alone procedures, or incorporated into catheter-or surgical-based procedures. For example, during a valve replacement surgery the TEE can be used to assess the valve function immediately before repair/replacement and immediately after. This permits revising the valve mid-surgery, if needed, to improve outcomes of the surgery.

Intracardiac echocardiography (ICE) is specialized form of echocardiography that uses catheters to insert the ultrasound probe inside the heart to view structures from within the heart. ICE is often used as a part of the cardiac procedure of crossing the interatrial septum with a transseptal puncture to permit catheter access from the right atrium to the left atrium; alternative access to the left heart would be retrograde through the aorta and across the aortic valve into the left ventricle.

ICE has the benefit over transthoracic echocardiography in that an operator who is performing a sterile procedure can also operate the ICE catheter and it is not limited to visibility problems that can arise with transthoracic or transesophageal echo. Though, there are image quality limitations due to size constraints of the probe being limited to a catheter.

ICE is often inserted through the femoral vein and into the right atrium. From the right atrium, visualization of the interatrial septum, all four cardiac chambers, all four valves, and the pericardial space (for an effusion) can be readily visualized. It can also be advanced across the atrial septum into the left atrium to visualize the left atrial appendage during left atrial appendage occlusion device deployment. Utilization of ICE imagery can be incorporated into the 3-D models built with electroanatomic mapping systems.

Electrical activity may also be detected using electrocardiography (an ECG or an EKG). An ECG may be a conventional 12-lead ECG, wherein ten electrodes are placed on the patient's limbs and on the surface of the chest. The overall magnitude of the heart's electrical potential is then measured from twelve different angles (“leads”) and is recorded over a period of time (usually ten seconds). In this way, the overall magnitude and direction of the heart's electrical depolarization is captured at each moment throughout the cardiac cycle. There are three main components to an ECG; the P wave, which represents depolarization of the atria, the QRS complex, which represents depolarization of the ventricles, and the T wave, which represents repolarization of the ventricles.

What is claimed is

1. A method for cycle length mapping, the method comprising:
recording electrical activity at a location on cardiac tissue as an electrogram;
identifying a characteristic deflection within the electrogram;
measuring times from the characteristic deflection to previous characteristic deflections;
modeling a distribution of the measured times; and
assigning a peak value of the distribution as a cycle length associated with the characteristic deflection at the location on the cardiac tissue.

2. The method of claim 1, wherein measuring step includes measuring back in time, on the electrogram, from the characteristic deflection, to a pre-determined number N of previous characteristic deflections on the electrogram.

3. The method of claim 1, wherein the modeled distribution comprises a kernel density estimation (KDE).

4. The method of claim 1, further comprising: identifying multiple characteristic deflections in each of multiple electrograms taken from corresponding locations on the cardiac tissue; and repeating the measuring, modeling, and assignment steps for the multiple characteristic deflections to create a cycle length map representing cycle lengths at the locations on the cardiac tissue.

5. The method of claim 4, wherein the cycle length map further presents cycle length variability at the locations on the cardiac tissue.

6. The method of claim 4, further comprising estimating, from the cycle length map, an effective refractory period for one or more of the locations on the cardiac tissue.

7. The method of claim 6, wherein the recording step comprises inserting a cardiac mapping catheter into a chamber of a heart of a patient and storing electrograms captured using a plurality of electrodes of an electrode array disposed on a distal portion of the catheter.

8. The method of claim 7, further comprising receiving signals from the electrode array at a computer system connected to the catheter, and operating the computer system to create and store the cycle length map.

9. The method of claim 7, wherein the plurality of electrodes is in the electrode array as pairs of electrodes, each pair in a stack that is substantially orthogonal to a surface of the cardiac tissue during use.

10. The method of claim 6, further comprising displaying a representation of the heart with the effective refractory period for the one or more of the locations on the cardiac tissue overlaid on the representation of the heart.

11. A system for cycle length mapping, the system comprising:
a cardiac mapping catheter with an electrode array disposed on a distal portion of the catheter, the electrode array comprising a plurality of electrodes; and
a computer system comprising a processor coupled to a memory subsystem having instructions stored therein executable by the processor to operate the electrode array to record electrical activity at a location on cardiac tissue as an electrogram when the distal portion of the catheter is inserted into a chamber of a heart of a patient, wherein the computer system is further operable to
identify a characteristic deflection within the electrogram;
measure times from the characteristic deflection to previous characteristic deflections;
model a distribution of the measured times; and
assign a peak value of the distribution as a cycle length associated with the characteristic deflection at the location on the cardiac tissue.

12. The system of claim 11, wherein the computer system measures, back in time from the characteristic deflection over the electrogram, to a pre-determined number N of previous characteristic deflections on the electrogram.

13. The system of claim 11, wherein the modeled distribution comprises a kernel density estimation (KDE).

14. The system of claim 11, further wherein the computer system is operable to:
identifying multiple characteristic deflections in each of multiple electrograms taken from corresponding locations on the cardiac tissue; and
repeat the measuring, modeling, and assignment steps for the multiple characteristic deflections to create a cycle length map representing cycle lengths at the locations on the cardiac tissue.

15. The system of claim 14, wherein the cycle length map further includes cycle length variability at the locations on the cardiac tissue.

16. The system of claim 14, further wherein the computer system is operable to estimate, from the cycle length map, an effective refractory period for one or more of the locations on the cardiac tissue.

17. The system of claim 16, wherein the memory subsystem stores therein electrograms captured using the plurality of electrodes of the catheter.

18. The system of claim 17, further comprising a processing device operably connected between the catheter and the computer system, wherein the processing device includes a at least one microprocessor that functions as a multiplexer, amplifier, or analog-to-digital (ADC) converter, wherein the processing device is a bedside instrument wired to the catheter that transmits data to the computer system.

19. The system of claim 17, wherein the plurality of electrodes is in the electrode array as pairs of electrodes, each pair in a stack that is substantially orthogonal to a surface of the cardiac tissue during use.

20. The system of claim 16, wherein the computer system is operable to display a representation of the heart with the effective refractory period for the one or more of the locations on the cardiac tissue overlaid on the representation of the heart.
